(19) United States
(12) Patent Application Publication
(10) Pub. No.: US 2003/0224398 A1
(43) Pub. Date: Dec. 4, 2003

(54) Purification of the leading front of migratory cells

(76) Inventor: Richard L. Klemke, San Diego, CA (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/365,961

(22) Filed: Feb. 12, 2003

Related U.S. Application Data
(60) Provisional application No. 60/356,893, filed on Feb. 13, 2002.

(51) Int. Cl. C12Q 1/00; C12Q 1/68; G01N 33/53; G01N 33/567; A61K 31/739; C12N 5/06

(52) U.S. Cl. 435/6; 435/4; 435/7.2; 435/317.1; 435/325; 514/54

(57) ABSTRACT

The invention relates generally to methods of modulating cell migration. Included in the methods of the invention are methods of identifying the state of a pseudopodium in cell migration and methods of inducing extension or retraction of a pseudopodia from a cell. The invention also relates to methods of screening for and identifying an agent effective in inducing extension or retraction of a pseudopodium and therefore affecting cell migration. Agents that can modulate cell migration are useful in treatment of conditions in which cell migration plays a role. Such conditions can include wound healing, angiogenesis, and metastasis of a disease from one location to another. Additionally, the invention provides methods of biochemically separating the pseudopodium of a cell from the remainder of the cell body and methods of determining the proteins present in the pseudopodium and cell body. The invention also includes a pseudopodium isolated by the methods of the invention.

### RELATED APPLICATIONS

This application claims priority under 35 U.S.C. § 119(e) to U.S. Ser. No. 60/356,893, filed Feb. 13, 2002, the entire content of which is incorporated herein by reference.

### GOVERNMENT SUPPORT

This invention was made in part with government support under Grant No. CA 78493-01 and Grant No. BCRP 6KB0046, awarded by the National Institutes of Health. The United States government may have certain rights in this invention.

### FIELD OF THE INVENTION

The present invention relates generally to methods and compositions for modulating cell migration, and more specifically to methods of purification of a dominant leading front, a pseudopodium, screening and purification of proteins contained in the pseudopodium and screening and purification of agents that affect cell migration.

### BACKGROUND INFORMATION

Directed cell movement or chemotaxis is exhibited during wound healing, angiogenesis, embryonic development, and immune function (Lauffenburger and Horwitz, 1996). This process is highly conserved, as prokaryotes and eukaryotes from Dictyostelium discoideum to human leukocytes exhibit the ability to sense and move in the direction of a chemoattractant (Parent and Devreotes, 1999; Jin et al., 2000; Servant et al., 2000).

Additionally, cell movement is seen in neuronal regeneration. Neuronal regeneration is the body's regeneration of cells in the brain and/or spinal cord resulting from a disorder. The regenerative process involves the movement of stem cells from the bone marrow to the injured area. Once in place, the stem cells assist in the repair of damaged neurons, generation of new neurons and blood vessels. Such disorders may occur as a result of head or spinal injury, such as a stroke, head injury or cerebral palsy or neurological diseases, such as Alzheimer's Disease, Parkinson's Disease, Multiple Sclerosis (MS) and Huntington's Disease. (WO 94/16718 (Fallon); U.S. Pat. No. 5,750,376 (Weiss)).

Recent evidence indicates that when eukaryotic cells encounter a chemoattractant gradient they respond by local activation and amplification of signals on the side facing the gradient (Parent et al., 1998; Meili et al., 1999; Jin et al., 2000; Servant et al., 2000). These signals facilitate localized actin polymerization leading to membrane protrusion in the direction of the gradient. The protrusion of a dominant leading pseudopodium (or lamellipodium) marks the first sign of morphological polarity with establishment of an anterior and posterior compartment, and occurs independently of cell body translocation or chemotaxis (Lauffenburger and Horwitz, 1996; Parent and Devreotes, 1999). Once a dominant pseudopodium is formed, cell movement commences in the direction of the gradient as the cell undergoes a cycle of membrane extension at the front and contraction at the rear.

Recent work has shown that integrin-cytoskeletal linkages, chemokine receptors, and actin regulatory proteins are spatially regulated in migratory cells (Ridley et al., 1992; Schmidt et al., 1993; Manes et al., 1999; Nobes and Hall, 1999; Eddy et al., 2000). Adhesive signals by integrins may also spatially localize to extending pseudopodia where they fine tune and maintain directional growth while suppressing retraction and detachment mechanisms (Smilenov et al., 1999; Kiosses et al., 2001; Laukaitis et al., 2001). Significant progress has also been made in determining the intracellular organization in migratory cells of pleckstrin homology (PH)-domain proteins, integrins, and the small GTPase Rac1 using GFP technology (Meili et al., 1999; Jin et al., 2000; Servant et al., 2000; Kraynov et al., 2001; Laukaitis et al., 2001).

Previous studies of pseudopodia have typically been done in individual cells, preventing molecular and biochemical evaluation of chemotactic signals and their spatio-temporal association with regulatory proteins and scaffolds. This is due to the lack of cellular material available for analysis, as well as the inability to specifically isolate the pseudopodium and cell body for biochemical comparison using previous techniques. The biochemical purification of the pseudopodium and cell body is necessary to understand the molecular detail of entire signaling networks and spatio-temporal mechanisms of regulation including protein translocation, activation/phosphorylation, and formation of complex multiprotein scaffolds.

### SUMMARY OF THE INVENTION

The present invention relates to methods for modulating cell migration, in particular by utilizing the morphological polarization of a cell undergoing migration and the biochemical separation of the same. The methods of the invention have been determined through analysis of the spatio-temporal localization and activation of cytoskeletal-associated signals in purified pseudopodia directed to undergo growth or retraction.

In one aspect, the invention provides a method of isolating a pseudopodium of a cell by placing a population of cells on a porous membrane and stimulating the cells with a chemoattractant, such that one of the cells is stimulated to extend a pseudopodium through the pores of the porous membrane. The result of such extension leaves a cell body on the opposite side of the membrane. The pseudopodia extending through the pores can then be removed and thereby isolated. In a further aspect, the proteins present in the pseudopodia can be identified. In still another aspect, the cell bodies can also be removed and isolated. In yet another aspect, the proteins of the cell bodies can be identified. In another aspect the invention provides a pseudopodium isolated by the above method.

The invention also provides a method of inducing extension of a pseudopodia from a cell by placing a population of cells on a porous membrane and stimulating the cells with a chemoattractant such that at least one cell is stimulated to extend a pseudopodium. Similarly, the invention provides a method of inducing retraction of a pseudopodia from a cell by placing a population of cells on a porous membrane and stimulating the cells with a chemoattractant such that at least one cell is stimulated to retract a pseudopodium.

In still another aspect, the invention provides a method of identifying an agent effective in inducing extension or retraction of a pseudopodium. This method of the invention is performed by placing a population of cells on a porous membrane and measuring the number of cells that have a pseudopodium extended through the pores of the porous membrane. The cells are then stimulated with an agent suspected of inducing extension or retraction of pseudopodia and the number of cells that have a pseudopodium extended through the pores is again measured. An increase in the number of cells extending a pseudopodium from the first measurement to the second is indicative of an agent effective in inducing extension of a pseudopodium. A decrease in the number of cells extending a pseudopodium from the first measurement to the second is indicative of an agent effective in inducing retraction of a pseudopodium. In yet another aspect the invention provides a method of modulating cell migration comprising administering an effective amount of an agent identified by the above method.

The invention, in another aspect, provides a method of modulating a cell migration-associated process by administering an effective amount of an agent identified by the method set forth above. In the method of the invention, the agent is effective in modulating the cell migration-associated process. Such cell migration-associated processes may include, but are not limited to cell and tissue development, wound healing, immune responses, angiogenesis, embryonic development, metastases, neuronal regeneration, stem cell migration, and inflammation.

In still another aspect, the invention provides a method of modulating cell migration by administering a composition that induces extension or retraction of a pseudopodium. By inducing extension or retraction of a pseudopodium, cell migration will be modulated.

Another aspect of the invention provides a method of identifying the state of a pseudopodium, i.e. whether the pseudopodium is extending or retracting, by measuring the level of Rac or Cdc42 in an isolated pseudopodium. In this aspect of the invention, an increase in Rac or Cdc42 activity in the pseudopodium is indicative a state of extension of the pseudopodium and a decrease of Rac or Cdc42 activity in the pseudopodium is indicative of a state of retraction of the pseudopodium.

In still another aspect, the invention provides a method of modulating cell migration comprising administration of an agent that regulates actin polymerization or depolymerization. In a related aspect, the invention provides a method of modulating cell migration comprising administration of an agent that regulates myosin contractility. In still another aspect, the invention provides a method of modulating cell migration comprising administration of an agent that regulates adhesive signals that facilitate attachment or detachment of a membrane to an extracellular matrix (ECM).

In another aspect, the invention provides a method of identifying proteins specifically expressed in the pseudopodium or specifically expressed in the cell body of a cell during a cell migration-associated process. The method is performed by identifying the proteins expressed in the pseudopodium during migration and identifying the proteins expressed in the cell body during migration and then comparing the proteins expressed in the pseudopodium to the proteins present in the cell body. Such a comparison allows identification of proteins similar to the pseudopodium and cell body. A difference in expression in the pseudopodium and the cell body is indicative of proteins specifically expressed in the pseudopodium or the cell body. In another aspect, the pseudopodium or the cell body may be isolated prior to identification of proteins specifically expressed therein. Once identified, the identified proteins may be used, for example, in a method of modulating a cell migration-associated process. Use of the proteins is not limited to the above use.

In still another aspect, the invention provides a method of identifying polynucleotides specifically expressed in the pseudopodium or specifically expressed in the cell body of a cell during a cell migration-associated process. This method is performed by identifying the polynucleotides expressed in the pseudopodium during migration and identifying the polynucleotides expressed in the cell body during migration. A comparison of the expression in the pseudopodium to the expression in the cell body is then performed. Comparison allows identification of expression of polynucleotides similar to the pseudopodium and cell body and a difference in expression is indicative of polynucleotides specifically expressed in the pseudopodium or the cell body.

### BRIEF DESCRIPTION OF THE DRAWINGS

FIG. 1 is a graphic illustration of the results of Example 2, for (a) NIH 3T3 and (b) COS-7 cells, showing that lysophosphatidic acid is a potent chemoattractant for both cell types.

FIG. 2 is an illustration of the results of Example 3, showing that cells extend pseudopodia through 3.0-μm pores toward an LPA gradient, but not toward a uniform concentration of LPA; a) shows the activity of NIH 3T3 cells, b) shows the activity of COS-7 cells, c) is the diffusion of 3H-LPA from the lower chamber to the upper chamber, d) is pseudopodia formation in COS-7 cells transfected with dominant negative Rac1, Cdc42 and an empty vector and e) is the result of examining the cells transfected as in d) for cell adhesion to collagen coated dishes in the presence and absence of LPA.

FIG. 3 is an illustration of the results of Example 4, showing the retraction of pseudopodia in NIH 3T3 and COS 7 cells upon removal of an LPA gradient; a) graph of retreating pseudopodia in NIH 3T3 cells after removal of LPA; b) graph of COS-7 pseudopodic extension in the presence of absence of antibodies to ανβ5 and β1 integrins; and c) graph of COS-7 pseudopodic retraction in the presence of absence of antibodies to ανβ5 and β1 integrins.

FIG. 4 shows the results of Example 6, the biochemical characterization of cytoskeletal-regulatory proteins in growing and retracting pseudopodia; a) proteins isolated from the cell body and pseudiapodia of NIH 3T3 cells during growth and retraction, as compared to controls; b) Western blotting of the proteins in a); c) examination of proteins for GTP-bound activated Rac, Cdc42 and Rho; d) western blot for tyrosine phosphorylation of NIH 3T3 cells in pseudopodia growth and retraction phase; e) total proteins isolated in d) analyzed with phosphorylation site-specific antibodies to FAK at tyrosines 397, 576 and 577; and f) proteins of a) were analyzed for tyrosine phosphorylation.

FIG. 5 is a series of illustrations showing the results of Example 7; a) western blots of Crk, CAS and FAK in NIH 3T3 cells with growing or retracting pseudopodium; b) pseudopodia protein in COS-7 cells transfected with an empty vector, a vector encoding CAS or a vector encoding Crk; c) pseudopodia protein in COS-7 cells transfected with a vector encoding CAS and Crk; d) pseudopodia protein in COS-7 cells transfected as in c) with pseudopodia extending or retracting; e) cell body and pseudopodia protein in COS-7 cells transfected as in c) examined for activated Rac or total Rac protein in whole cell lysates.

FIG. 6 is an illustration of the Rac regulation of CAS/Crk complexes in COS-7 cells, as set forth in Example 8.

FIG. 7 is a proposed model for the role of CAS/Crk coupling in regulation of Rac-mediated pseudopodial dynamics.

### DETAILED DESCRIPTION OF THE INVENTION

The present invention provides methods for modulating cell migration through purification of pseudopodia involved in cell migration. The invention is based on the findings that that pseudopodia can be purified in the process of growth or retraction, that pseudopodia extension and retraction require Rac activation and deactivation, respectively, through spatial assembly and disassembly of a CAS/Crk protein scaffold. Also provided are agents that can be used to modulate cell growth and retraction and methods for identifying such agents.

As cell migration is involved in processes, including but not limited to cell and tissue development, wound healing, immune responses, angiogenesis, embryonic development, metastases, neuronal regeneration, stem cell migration and inflammation, for example, modulation of cell migration also allows modulation or regulation of these processes. As such, these processes are referred to herein as “cell migration-associated processes.”

“Chemotaxis” as used herein refers to the directed movement of a cell. Such movement is in response to a substance exhibiting chemical properties which can attract or repel the cell. For example, a cell can undergo chemotaxis to the site of a wound by being attracted by chemicals released by damaged cells or by chemicals produced by bacteria in a cut or scratch. A “chemoattractant” as used herein is a substance that attracts the cell.

The initial step of chemotaxis involves morphological polarization of the cell in response to the chemoattractant, with formation of a leading front, such as a pseudopodium, and a rear compartment. “Pseudopodium” translates from Latin as a “false foot,” but is actually a temporary, retractable extension of a cell's cytoplasm. The pseudopodia discussed herein are used to advance the cell position.

As a pseudopodium extends and attaches to a substrate, the cytosol of the cell initially remains in the rear of the cell, such that the cell has two distinct regions, the pseudopodium and the cell body, but the cytosol then flows forward into the pseudopodium, carrying the bulk of the cell with it and continues to flow forward. The pseudopodium retracts into the trailing edge of the cell. The process repeats as the cell continues to be attracted to the chemoattractant.

Chemotaxis requires cells to sense the direction and proximity of a chemoattractant. This requires activation of localized signals and actin polymerization on the cell membrane facing the gradient. The term “gradient” as used herein refers to a concentration gradient, which is a change in concentration of over a distance, such as a gradient across a membrane.

“Chemokinesis,” as opposed to chemotaxis, as used herein refers to random cell migration, or persistent cell movement in the absence or presence of a uniform concentration of chemokine (Lauffenburger and Horwitz, 1996).

To determine whether lysophosphatidic acid (LPA) (Fukushima et al., 2001) and insulin induced directed or random cell migration, NIH 3T3 and COS-7 cells were examined for cell migration through 8.0-μm porous membranes in the presence of either a gradient or uniform concentration of these chemokines (Example 2). Only cells exposed to an LPA gradient were induced to migrate, indicating that LPA is a true chemoattractant and does not facilitate random cell migration. LPA-induced chemotaxis was dose dependent and reached a maximum with 100-200 ng/ml. In contrast, insulin promoted random migration, but was a poor chemoattractant for both cell types. Therefore, in one aspect of the invention, a chemoattractant such as LPA can be used to induce cell chemotaxis.

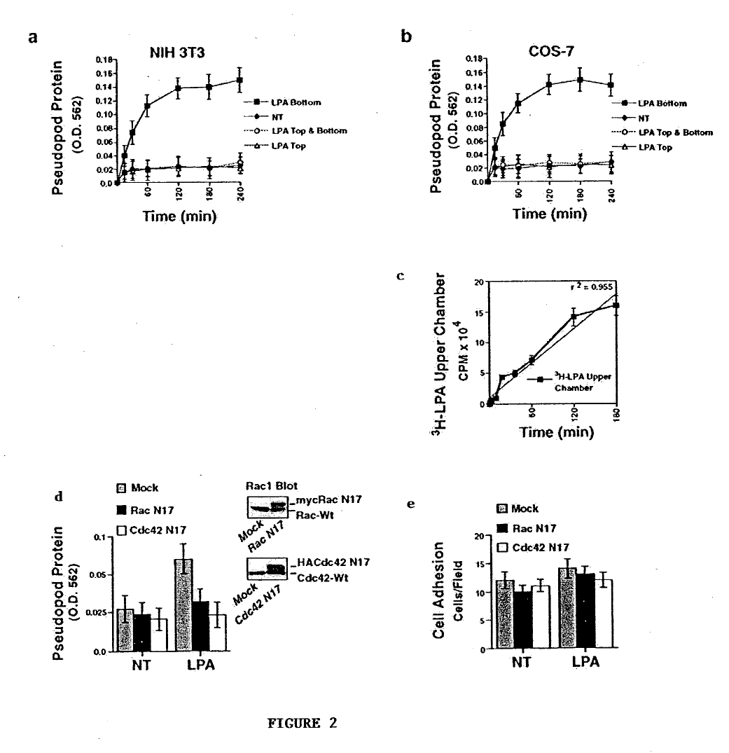

By the present invention, it has been determined that extension of a pseudopodium is independent of cell body translocation. When cells respond to a chemoattractant gradient they extend pseudopodia in the direction of the chemoattractant before cell translocation. The formation of a dominant leading pseudopodium establishes cell polarity and the future direction of chemotaxis. Cells exposed to a gradient, but not a uniform concentration of LPA, were found to extend pseudopodia through small pores specifically in the direction of the gradient (Example 3). Using a confocal microscope sequentially focused at the upper and lower membrane surface, it was seen that >90% of the cells polarized by extending pseudopodia in response to an LPA gradient. Extension of pseudopodia through 3.0-μm pores was first detected 10-15 min after exposure to the LPA gradient and proceeded linearly for 60-90 min (Example 3). Formation of the LPA gradient under these conditions was steep and linear for at least 3 hours (FIG. 2C). In addition, nuclei were not detected (by DAPI staining) on the lower surface of the membrane, but were restricted solely to the cell body on the upper surface, indicating that only pseudopodia protrude through the pores. Additionally, pseudopodia extension was prevented in cells expressing dominant negative mutants of the small GTPase Rac1 (RacN17) or Cdc42 (Cdc42N17) (Ridley et al., 1992) (FIGS. 2D and 2E). Rac and Cdc42 are well known in the art to control cell polarity through regulation of actin protrusive processes at the leading front of migratory cells (Allen et al., 1998; Nobes and Hall, 1999; Etienne-Manneville and Hall, 2001). Together, these findings establish that cells obtain polarity by extending leading pseudopodia through 3.0-μm pores toward a gradient of LPA, or another chemoattractant, in a Rac and Cdc42 dependent manner, independent of cell body translocation.

In one aspect the invention provides a method of inducing extension of a pseudopodia from a cell by placing a population of cells on a porous membrane and stimulating the cells with a chemoattractant such that at least one cell is stimulated to extend a pseudopodium. The chemoattractant can be, but is not limited to, LPA.

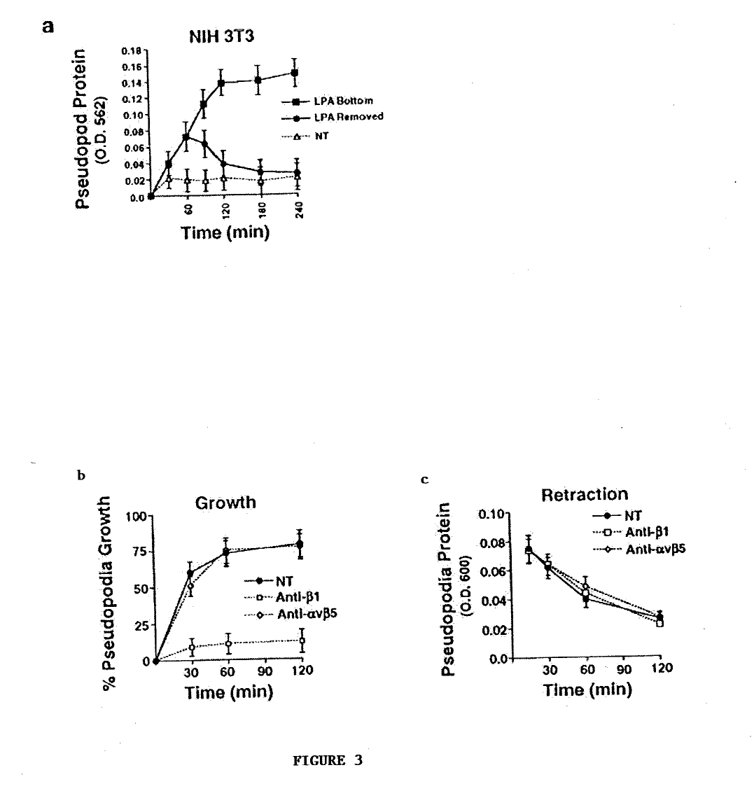

As a cell migrates, it is a continual process of pseudopodia extension and retraction. However, upon removal of the chemoattractant, the pseudopodia retract and cell polarity is lost. In Example 4, set forth below, cells were induced to extend pseudopodia toward an LPA gradient for 60 min. The gradient was then removed from the lower chamber and pseudopodia retraction was examined for various times. Loss of the LPA gradient was sufficient to reverse the polarized phenotype and induce pseudopodia retraction (FIG. 3A). Confocal imaging and time-lapse determination revealed that pseudopodia retraction began within 5 minutes of removing the gradient and proceeded for 2 hours. No new pseudopodia extensions were observed during this time period, and >80% of the cells displayed pseuodopodia retraction under these conditions. Restoring the LPA gradient to these cells inhibited the retraction process and induced pseudopodia growth. Therefore, cells retract their pseudopodia protrusions and lose cell polarity upon removal of the chemoattractant gradient. In one aspect of the invention, pseudopodia extension and retraction can be controlled by the presence of a chemoattractant.

In another aspect, the invention provides a method of inducing retraction of a pseudopodia from a cell by placing a population of cells on a porous membrane and stimulating the cells with a chemoattractant such that at least one cell is stimulated to retract a pseudopodium. Such methods of stimulating retraction can include, but are not limited to applying a uniform concentration of chemoattactant to the population of cells. Such chemoattactants can include, but are not limited to, LPA and insulin.

In still another aspect, the invention provides a method of identifying an agent effective in inducing extension or retraction of a pseudopodium. This method of the invention is performed by placing a population of cells on a porous membrane and measuring the number of cells that have a pseudopodium extended through the pores of the porous membrane. The cells are then stimulated with an agent suspected of inducing extension or retraction of pseudopodia and the number of cells that have a pseudopodium extended through the pores is again measured. An increase in the number of cells extending a pseudopodium from the first measurement to the second is indicative of an agent effective in inducing extension of a pseudopodium and a decrease in the number of cells extending a pseudopodium from the first measurement to the second is indicative of an agent effective in inducing retraction of a pseudopodium. In a further aspect of the method, the pseudopodia that are extended are isolated and the proteins present therein are determined. In one aspect the agent increases the number of cells extending a pseudopodium. In still another aspect the agent stimulates at least about 90% of the cells to extend a pseudopodium. In another aspect the agent decreases the number of cells extending a pseudopodium. In yet another aspect the invention provides a method of modulating cell migration comprising administering an effective amount of an agent identified by the above method.

The invention also provides a method of modulating cell migration by administration of a composition that induces extension or retraction of a pseudopodium, thereby modulating cell migration.

In still another aspect, the invention provides a method for modulating cell migration-associated processes that are dependent upon cell migration by modulating the cell migration itself. The invention provides a method of modulating cell migration-associated processes by administering an effective amount of an agent identified by the above methods. Such cell migration-associated processes can include, but are not limited to cell and tissue development, wound healing, immune responses, angiogenesis, embryonic development, metastases, neuronal regeneration, stem cell migration and inflammation. Accordingly, it is an object of the invention to find agents, compositions or drugs that are useful in modulating cell migration-associated processes. Such agents may be used to target proteins or polynucleotides identified by the methods of the invention.

Recent evidence indicates that integrin adhesion receptors play a critical role in facilitating and maintaining directional growth of pseudopodia on extracellular matrix (ECM) proteins (Bailly et al., 1998; Kiosses et al., 2001; Laukaitis et al., 2001). In Example 4, the necessity of integrins for pseudopodia extension on a collagen substrate was shown. COS-7 cells were allowed to attach to collagen-coated membranes for 2 hours and then exposed to an LPA gradient in the presence of function-blocking antibodies to β1 integrins, which facilitates attachment of these cells to collagen (Cho and Klemke, 2000). The anti-β1 antibody prevented pseudopodia growth on collagen, whereas control antibodies to the vitronectin receptor vβ5 present on these cells did not (FIG. 3B). The β1 blocking antibodies specifically prevented pseudopodia extension and did not cause detachment of the cell body from the substratum. The anti-β1 antibodies did not alter pseudopodia retraction, indicating that formation of new adhesion contacts were not necessary for the retraction process per se (FIG. 3C). These findings demonstrate that integrins are necessary for pseudopodia growth on the ECM, and provide additional evidence that new pseudopodia do not extend through the pores after the gradient is removed, as this would depend on new integrin contacts.

The invention demonstrates that pseudopodia growth and retraction is a dynamic process involving changes in focal adhesions and the actin cytoskeleton. In another aspect, the invention provides a method of biochemically separating the cell into its leading pseudopodium and cell body for examination and protein analysis. Such isolation allows a direct examination of cytoskeletal components as well as complex signaling pathways that control cell polarity. By utilizing the discovery that that cells polarize by extending pseudopodia through 3.0-μm pores, only in the direction of a chemoattractant gradient, separation and purification of the pseudopodium and cell body is possible.

Therefore, the invention provides a method of isolating a pseudopodium of a cell by placing a population of cells on a porous membrane and stimulating the cells with a chemoattractant, such that one of the cells is stimulated to extend a pseudopodium through the pores of the porous membrane. The result of such extension leaves a cell body on the opposite side of the membrane. The pseudopodia extending through the pores can then be removed and thereby isolated. The pseudopodia can be removed with a cotton swab or by any other method known to those of skill in the art. The porous membrane utilized in the method can be, but is not limited to a porous polycarbonate membrane. In another aspect the invention provides a pseudopodium isolated by the above method.

In a further aspect, the proteins or polynucleotides expressed in the pseudopodia can be identified, screened, compared and/or isolated. In still another aspect, the cell bodies can also be removed and isolated. In yet another aspect, the proteins or polynucleotides expressed in the cell bodies can be identified.

The term “identify” as used herein is used to refer to establishing the identity of a protein or polynucleotide expressed in a cell or a portion thereof, i.e. the pseudopodium or cell body. Establishing the identity means determining the distinguishing characteristics of the protein or polynucleotide. In the method of the invention, identification of a protein or polynucleotide expressed in a cell or a portion thereof allows identification of a target, which may be utilized for modulation of cell migration and cell migration-associated processes.

In one aspect, the invention provides a method of identifying proteins specifically expressed in the pseudopodium or specifically expressed in the cell body of a cell. In particular, the method is utilized to identify the proteins when the cell is undergoing a cell migration-associated process. In the method, the proteins expressed in the pseudopodium during migration are identified and the proteins expressed in the cell body during migration are identified. The proteins expressed in each are compared, and the comparing allows identification of proteins similar to the pseudopodium and cell body. A difference in expression is indicative of proteins specifically expressed in the pseudopodium or the cell body. In yet another embodiment, the pseudopodia are isolated prior to identifying the proteins. In still another embodiment, the cell bodies are isolated prior to identifying the proteins. The comparing of the proteins expressed in the pseudopodium and the proteins expressed in the cell body may be performed by any method known to those of skill in the art. Methods of comparing may include, but are not limited to brute force mass spectrometry and two dimensional gel electrophoresis.

In another aspect the invention provides a method of modulating a cell migration-associated process comprising administering an effective amount of an agent targeted to modulate expression of a protein identified by the method set forth above. Such a cell migration-associated process may include, but is not limited to cell and tissue development, wound healing, immune responses, angiogenesis, embryonic development, metastases, neuronal regeneration, stem cell migration, and inflammation

In still another aspect the invention provides a method of identifying polynucleotides specifically expressed in the pseudopodium or specifically expressed in the cell body of a cell. In particular, the method is utilized to identify the polynucleotides when the cell is undergoing a cell migration-associated process. In the method, the polynucleotides expressed in the pseudopodium during migration are identified and the polynucleotides expressed in the cell body during migration are identified. The polynucleotides expressed in each are compared, and the comparing allows identification of expression of polynucleotides similar to the pseudopodium and cell body. A difference in expression is indicative of polynucleotides specifically expressed in the pseudopodium or the cell body. In yet another embodiment, the pseudopodia are isolated prior to identifying the polynucleotides. In still another embodiment, the cell bodies are isolated prior to identifying the polynucleotides.

The term “screening” as used herein refers to methods for identifying an agent or protein of interest. Where an agent is to be screened, it is chosen from agents potentially effective in inducing extension or retraction of a pseudopodium, and therefore useful in modulation of cell migration. Where a protein is to be screened, it is selected from all proteins present in an isolated portion of a cell undergoing cell migration. For example, the method of screening proteins can include isolating the pseudopodium of a cell undergoing cell migration and identifying which proteins present therein possess characteristics of interest. Preferably, the method permits the identification of an agent or protein of interest among one or more agents or proteins. The term “screening” describes what is, in general, a two-step process in which one first determines which agent or protein does or does not express a desired characteristic and then physically separates the cells having the desired characteristic.

Screening can include both classical screening, whereby expression of a nucleic acid results in a phenotype that can be identified (for example by having a colony with the nucleic acid of interest change color, fluoresce, or luminesce), and can also include classical selection, where typically the phenotype to be identified is growth on selective media. By “selective” is meant media on which the host strain will not grow or grows poorly, but that strains with the nucleic acid of interest will grow in a manner which can be readily distinguished from host strain growth by methods well-known in the art. In some forms of screening, identification and physical separation are achieved simultaneously. For example, identification and separation of a peptide that affects extension of a pseudopodium in a cell can be accomplished by selecting cells undergoing pseudiopodium extension.

The term “compared,” as used herein is used in the examination of a group of proteins or polynucleotides. The proteins or polynucleotides are examined for characteristics or qualities possessed by each that may share similarities or lack similarities among the proteins or polynucleotides. In one aspect of the invention, proteins identified in the pseudopodium of a cell undergoing migration or a cell migration-associated process are compared to proteins identified in the cell body of the cell to determine which proteins are involved in the migration or cell migration-associated process. Such comparisons may be performed by any method known to those of skill in the art. In one aspect the comparison is performed by brute force mass spectrometry or by two dimensional gel electrophoresis.

Comparison is therefore integrally related to identification. Through the methods of the invention, proteins or polynucleotides can be identified which can then be targeted in order to control cell migration or cell migration-associated processes. Cell migration-associated processes may include, but are not limited to cell and tissue development, wound healing, immune responses, angiogenesis, embryonic development, metastases, neuronal regeneration, stem cell migration, and inflammation.

The term “isolated,” when applied to a nucleic acid or protein, means altered “by the hand of man” from the natural state and denotes that the nucleic acid or protein is essentially free of other cellular components with which it is associated in the natural state. Purity and homogeneity are typically determined using analytical chemistry techniques such as polyacrylamide gel electrophoresis or high performance liquid chromatography. The term “purified” denotes that a nucleic acid or protein gives rise to essentially one band in an electrophoretic gel. Particularly, it means that the nucleic acid or protein is at least about 50% pure, at least about 85% pure, or at least about 99% pure.

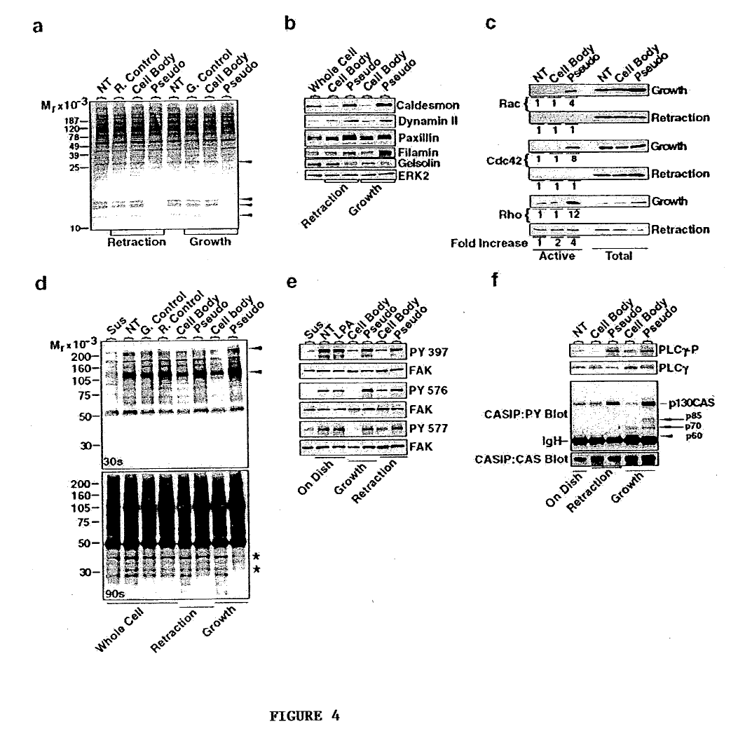

One example of such isolation is set forth in Example 5. Another example of isolation is Example 6. In Example 6, cells were allowed to extend pseudopodia for 60 minutes toward a chemoattractant gradient or the gradient was removed and cells were allowed to retract for 30 minutes. At these times, the chemoattractant gradient as well as pseudopodia growth and retraction are linear, as can be seen in FIG. 2. The cell body on the upper surface of the membrane was manually removed and the pseudopodia on the undersurface extracted with detergent. The cell body was purified in a similar manner except that pseudopodia on the lower surface were manually removed and the cell body on the upper surface extracted with detergent. The total profile of proteins isolated from the cell body and pseudopodium was normalized and then analyzed by one-dimensional SDS-PAGE. Abundant proteins were similar in the cell body and pseudopodium, indicating that the samples were normalized appropriately (FIG. 4A). However, nuclear histones were clearly absent from the pseudopodium, providing additional evidence for the selective purification of this structure (14-18- and 30-kD proteins) (FIG. 4A).

The isolated pseudopodia and cell bodies were also analyzed for cytoskeletal-associated proteins and tyrosine phosphorylation in growing and retracting pseudopodia. Caldesmon, dynamin II, paxillin, and filamin were all substantially increased in the pseudopodium (FIG. 4B) (Ridley et al., 1992; Helfman et al., 1999; Ohta et al., 1999; McNiven et al., 2000; Laukaitis et al., 2001). In contrast, the actin-severing protein gelsolin was restricted to the cell body proper and was not present in the pseudopodium, whereas extracellular-regulated kinase (ERK) 2 did not show a spatial change in polarized cells (Azuma et al., 1998).

Rho, Rac, and Cdc42 showed significantly increased activity in the extending pseudopodium compared with the cell body (FIG. 4C). Associated with the increased GTPase activity was increased Rho and Rac, but not Cdc42 protein levels, in the pseudopodium. Retracting pseudopodia showed decreased Rho, Rac, and Cdc42 activity, as well as decreased Rho and Rac protein levels. However, whereas Rho activity was clearly decreased during retraction, a notable amount of Rho activity remained in the pseudopodium under these conditions (FIG. 4C). There was also a small increase in Rho activity in the body of retracting cells. It is known in the art that Rho may play an important role in both growth and retraction processes through its ability to regulate Rac and Cdc42 activity as well as the actin/myosin contractile machinery (Schmitz et al., 2000). Pseudopodia isolated after 15 and 90 minutes of growth or 15 and 90 minutes of retraction showed identical results. During LPA-induced pseudopodia growth there is no change in Rho, Rac, or Cdc42 activity in the cell body relative to the nontreated whole cell group. Thus, it is clear that the cell body on the upper surface does not simultaneously extend pseudopodia, as this would also lead to increased GTPase activity in the upper compartment. Together, these findings demonstrate the biochemical purification of pseudopodia and the spatial segregation of cytoskeletal regulatory proteins in polarized cells.

Another aspect of the invention provides a method of identifying the state of a pseudopodium, i.e. whether the pseudopodium is extending or retracting, by measuring the level of Rac or Cdc42 in an isolated pseudopodium. In this aspect of the invention, an increase in Rac or Cdc42 activity in the pseudopodium is indicative a state of extension of the pseudopodium and a decrease of Rac or Cdc42 activity in the pseudopodium is indicative of a state of retraction of the pseudopodium.

Establishment of a leading pseudopodium requires spatial regulation of actin polymerization and formation of focal adhesions, which is associated with tyrosine phosphorylation of cellular proteins (Lauffenburger and Horwitz, 1996; Aplin et al., 1998). Therefore, in the present invention, the phosphotyrosine protein profile in the cell body and pseudopodium of cells polarized towards a chemoattractant gradient were examined. Phosphoproteins of 220, 125-130, 100, and 70 kD were present in purified pseudopodia and either absent or dephosphorylated in the cell body (FIG. 4D). These proteins were dephosphorylated in suspension cells, indicating that their phosphorylation is dependent on cell adhesion to the ECM. In contrast, phosphoproteins of 74 and 28 kD were present in the cell body and were either absent or dephosphorylated in the pseudopodium. Phosphotyrosine proteins appeared similar under growth and retraction conditions except for a prominent 40-kD protein that was phosphorylated only in pseudopodia undergoing retraction, but not growth, suggesting a role for this protein in the retraction process (FIG. 4D).

Analysis of the focal adhesion associated proteins FAK, CAS, and PLC-1 revealed that these p120-130-kD phosphoproteins were strongly activated/phosphorylated during pseudopodia growth, as well as retraction, although there can be a small decrease in FAK 576 and 577 phosphorylation during retraction (FIGS. 4E and 4F). The relative levels of FAK, CAS, and PLC-1 were similar in the pseudopodium and cell body of polarized cells during growth and retraction. Thus, the protein level and tyrosine phosphorylation of these signals do not change significantly (FIGS. 4E and 4F), even though pseudopodium growth/retraction involves dynamic focal contact remodelling and actin cytoskeletal changes. These findings suggest that general dephosphorylation or loss of focal adhesion-associated signals is not the primary mechanism responsible for pseudopodium retraction and detachment from the substratum. Therefore, the process of pseudopodia retraction is not simply the reverse of the signaling processes that mediate growth. This is significantly different than cell detachment from the ECM, which is accompanied by complete dephosphorylation and inactivation of FAK, CAS, and PLC-1 (Aplin et al., 1998). Assembly and disassembly of a CAS/Crk complex controls Rac localization and activity, which is necessary for pseudopodia growth and retraction, respectively.

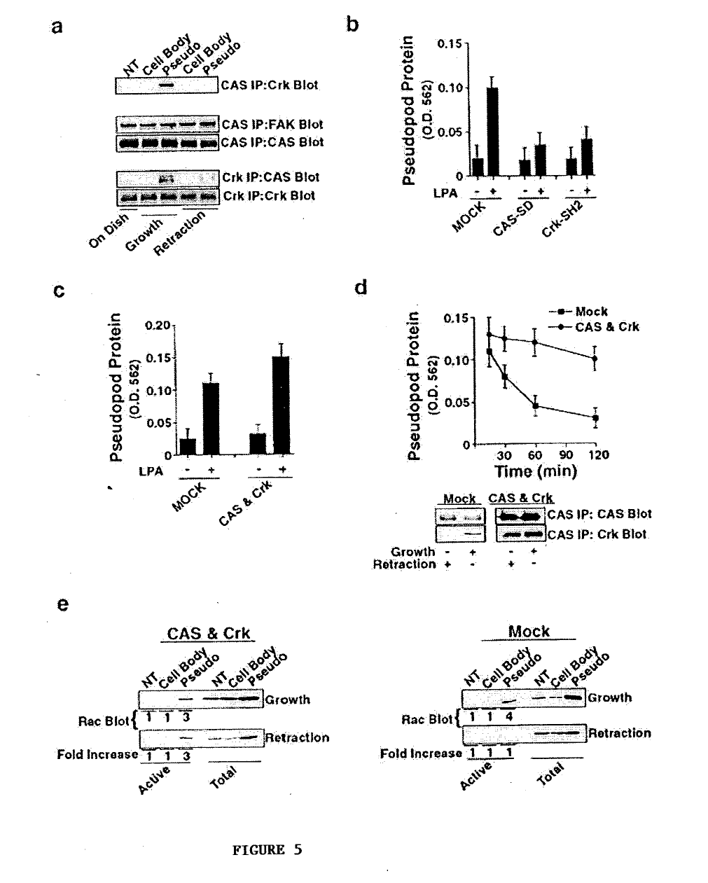

The role of tyrosine phosphorylation of these and other cytoskeletal regulatory proteins therefore may be that these proteins assemble specific signaling scaffolds consisting of unique kinases, substrates, and effector proteins that regulate this process. Formation of a FAK/CAS/Crk protein complex, as the spatio-temporal assembly of this scaffold and its role in mediating cell migration are poorly understood (Klemke et al., 1998). In Example 7, and FIG. 5, it is seen that Crk strongly associated with CAS in growing but was significantly reduced in retracting pseudopodia (FIG. 5A). The interaction was specific to the pseudopodium, as CAS isolated from the cell body of polarized cells did not show Crk binding. In contrast, FAK, which binds to the src-homology (SH)3 domain of CAS, showed no difference in binding to CAS under these conditions (FIG. 5A) In addition, several tyrosine-phosphorylated proteins that coimmunoprecipitated with CAS in growing, but not retracting pseudopodia were observed (FIG. 4F). In contrast, a 60-kD phosphoprotein associated with CAS only in the cell body, but not the pseudopodium was also observed. Denaturation of cellular proteins before CAS immunoprecipitation prevented the association of these proteins with CAS, indicating that the interaction is specific and not related to antibody cross-reactivity. Thus, CAS/Crk complexes assemble and disassemble in a highly spatial manner during pseudopodia extension and retraction.

Cell adhesion to the ECM promotes Crk binding via its SH2 domain to phosphotyrosine residues present in the substrate domain (SD) of CAS, whereas cell detachment causes complete dephosphorylation of CAS and disassembly of CAS/Crk complexes (Vuori et al., 1996; Klemke et al., 1998). The overall level of protein and tyrosine phosphorylation of CAS does not change during pseudopodium retraction and detachment from the ECM, but Crk binding does. Therefore, assembly and disassembly of CAS/Crk in the pseudopodium is tightly regulated through phosphorylation/dephosphorylation of a specific subset of tyrosine residues present in CAS. Alternatively, phosphorylation of the regulatory tyrosine 221 of Crk, which prevents CAS/Crk coupling in cells, may regulate this process (Kain and Klemke, 2001). However, significant changes in tyrosine phosphorylation of Crk during pseudopodia growth and retraction were not detected. Therefore, tyrosine phosphorylation of the substrate domain of CAS may be a critical event involved in CAS/Crk coupling and pseudopodium formation. In Example 7 pseudopodia formation in cells expressing CAS with its substrate domain deleted (CAS-SD) was examined. CAS-SD or Crk with a mutated SH2 domain (Crk-SH2) serve as dominant negative proteins preventing CAS/Crk coupling and Rac activation in motile cells (Klemke et al., 1998). Expression of CAS-SD or Crk-SH2 prevented pseudopodia extension in response to an LPA gradient (FIG. 5B). These findings demonstrate that CAS/Crk coupling is necessary for cell polarization and extension of a leading pseudopodium.

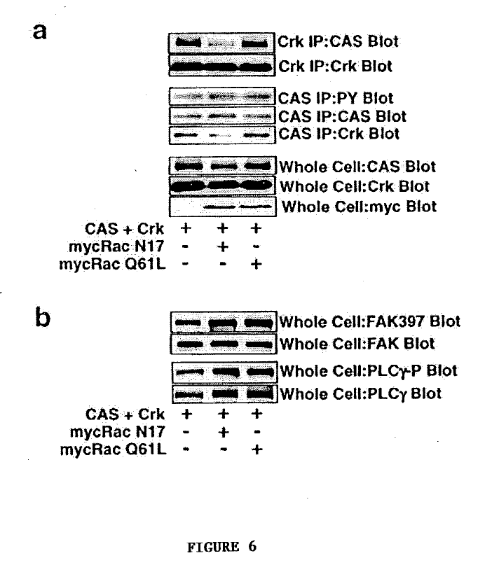

To investigate whether CAS/Crk coupling could enhance pseudopodia extension, full-length CAS and Crk were exogenously expressed in cells. However, these cells did not show a significant change in pseudopodia extension in response to an LPA gradient, suggesting that these molecular signals are not limiting to the process of gradient sensing and chemotaxis and that an additional component(s) is required to mediate this process (FIG. 5C). This is different from haptotaxis migration, as exogenous CAS/Crk coupling in cells is sufficient to facilitate strong migration as well as pseudopodia extension toward an adhesive gradient of ECM protein in the absence of a soluble chemoattractant (Klemke et al., 1998). Surprisingly, whereas increased CAS/Crk coupling in these cells did not impact pseudopodia extension, it did prevent membrane retraction, when the gradient was removed (FIG. 5D). In this case, CAS/Crk complexes were not disassembled, and the activation and translocation of Rae into pseudopodia were persistent (FIGS. 5D and 5E). These findings suggest that sustained Rae activity sends a positive feedback signal to upstream kinases/phosphatases that regulate CAS/Crk coupling and pseudopodium extension. In Example 8, CAS/Crk coupling in cells expressing dominant negative RacN17 was examined. Inhibition of Rae activity in cells significantly decreased CAS/Crk coupling, which was independent of changes in FAK and PLC-1 activity (FIGS. 6A and 6B). Moreover, the inhibition of CAS/Crk coupling was independent of changes in overall CAS tyrosine phosphorylation. Expression of activated RacQ61L in cells did not alter CAS/Crk coupling, indicating that this complex was maximally activated in these cells (FIG. 6A). Therefore, Rac can operate upstream to specifically regulate assembly of CAS/Crk complexes in cells. This supports the idea that Rac activation serves as a positive feedback signal to modulate CAS/Crk coupling in migratory cells. Together, these results demonstrate that assembly of a CAS/Crk complex is important for pseudopodia extension, whereas disassembly facilitates retraction through deactivation of Rac and its export from this cellular structure.

In still another aspect the invention provides methods of quantifing as well as visualizing pseudopodia in living cells using confocal microscopy and time-lapse imaging. In yet another aspect the invention provides a method of analysis of signaling dynamics of growing and retracting pseudopodia in live cells.

By the methods of the invention, it was demonstrated that CAS and Crk are specifically assembled during pseudopodia growth and then disassembled during retraction. This process occurred without apparent change in the overall level of CAS tyrosine phosphorylation or FAK activation, which is an upstream activator of CAS (Vuori et al., 1996; Tachibana et al., 1997). One explanation is that Crk couples only to a specific subset of phosphotyrosine residues present in the substrate domain of CAS, which changes during pseudopodia growth and retraction. CAS/Crk association is mediated through the binding of the SH2 domain of Crk to phosphotyrosine residues present in the substrate domain of CAS (Matsuda et al., 1993). In fact, there are 15 tyrosine residues in this region of CAS that correspond to potential SH2 binding motifs, 9 of which conform to the Crk SH2 recognition sequence YD(V/T)P (Klemke et al., 1998). Alternatively, regulation may occur through serine phosphorylation of CAS (Ma et al., 2001) or phosphorylation of the regulatory tyrosine 221 of Crk, which prevents CAS/Crk coupling in cells (Kain and Klemke, 2001). However, the latter is unlikely as no significant change in Crk tyrosine phosphorylation was detected. The upstream and downstream components that modulate the assembly/disassembly of this molecular scaffold in the pseudopodium are not yet clear, but likely candidates include c-src, PTP-PEST, and PTP-1B (Garton et al., 1996; Liu et al., 1996; Vuori et al., 1996).

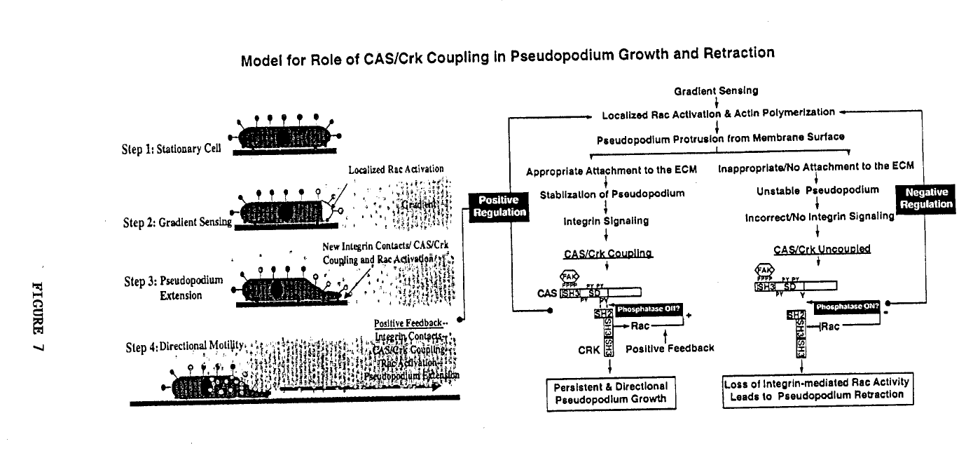

The active translocation of Rac to the pseudopodium also implies that there is an import/export mechanism that controls Rac transport to the different poles of migratory cells. It is unlikely that CAS and Crk are directly involved in this process, as these proteins do not appear to translocate in and out of growing and retracting pseudopodia. Rather, it is more likely that assembly of CAS/Crk is important for maintaining localized Rac activity within the pseudopodium. For example, it may be that when cells encounter a chemoattractant gradient, Rac (and associated regulatory proteins) translocates to the side of the membrane facing the gradient as previously described for PH domain proteins like Akt (Servant et al., 2000) (FIG. 7). The localized Rac activity then induces actin polymerization leading to protrusion of a pseudopodium from the cell surface and recruitment of high-affinity integrins to this region (Kiosses et al., 2001). Interestingly, protrusion of the pseudopodium from the membrane surface is independent of integrin ligation and attachment to the ECM (Bailly et al., 1998). This clearly places Rac activation and actin protrusive mechanisms as an early response upstream of integrin ligation and CAS/Crk assembly. However, pseudopodia attachment to the ECM is critical to stabilize the protrusive structure, as protruding membranes that do not contact the ECM readily retract back to the cell body (Bailly et al., 1998). Therefore, it is likely that during pseudopodia attachment to the substratum, integrin activation and focal complex formation play a central role in driving assembly of CAS/Crk, leading to sustained Rac activation and persistent membrane protrusion. The methods of the invention suggest that the persistent Rac activity then serves as a positive input to maintain a high level of CAS/Crk coupling at the leading front (FIG. 7).

FIG. 7 illustrates a proposed model for the role of CAS/Crk coupling in regulation of Rac-mediated pseudopodial dynamics. Step 1 involves attachment of stationary cells to the underlying ECM through integrin receptors. In step 2, cells are then exposed to a soluble gradient of growth/factor or chemokine. This activates cell surface chemoattractant receptors leading to activation and amplification of Rac signaling events on the side facing the gradient. In step 3, Rho, Cdc42, and Rac then regulate localized actin dynamics as well as force requirements leading to membrane protrusion in the direction of the gradient. This process is independent of actual cell body translocation or chemotaxis and marks the first sign of morphological polarity with establishment of a dominant leading pseudopodium and posterior compartment. Importantly, evidence indicates that the initial protrusion of a pseudopodium at the cell surface is independent of integrins and the ECM contacts. However, integrins do play a critical role in pseudopodial dynamics by tethering the extending membrane to the substratum, which initiates the molecular coupling of CAS and Crk. CAS/Crk coupling in turn mediates Rac activity leading to sustained and directional pseudopodium growth. A pseudopodium that does not attach to the ECM rapidly retracts back to the cell body. Once a dominant pseudopodium is formed, step 4 is cell movement commencement in the direction of the gradient as the cell undergoes repeated cycles of membrane protrusion, adhesion to the ECM, and CAS/Crk/Rac activation. Importantly, these findings suggest that sustained Rac activity then provides a positive feedback signal to maintain CAS/Crk coupling and membrane extension at the leading front as the cell moves on the ECM. On the other hand, loss of the chemoattractant gradient or inappropriate contact with the ECM terminates pseudopodium extension, turning off CAS/Crk/Rac activity and chemotaxis. Therefore, in this model integrin ligation events at the leading front of the extending membrane cooperate with chemoattractant receptors to fine tune and maintain directional growth, while suppressing retraction mechanisms through regulation of CAS/Crk and Rac.

In contrast, withdrawal of the chemotactic signal terminates membrane extension and focal adhesion formation resulting in uncoupling of the CAS/Crk/Rac signaling module leading to pseudopodium retraction. Interestingly, the disassembly of this complex and membrane retraction occur independently of changes in FAK and PLC-1 activity, and CAS tyrosine phosphorylation. This suggests that the Rac feedback pathway specifically regulates CAS/Crk coupling and does not generally block upstream integrin signaling processes. The Rac feedback loop may target a phosphatase that dephosphorylates a specific tyrosine residue(s) in the substrate domain of CAS, which facilitates Crk binding. Apparently, integrin and cytoskeletal signals continue to play a prominent role during pseudopodium retraction by assembling and regulating new protein scaffolds that mediate this process. Pseudopodium retraction is not the simple reversal of the adhesive-signaling processes that facilitate growth.

By the present invention, several lines of evidence for the biochemical purification of pseudopodia are presented. First, >90% of cells polarize by extending pseudopodia through 3.0-μm pores in the direction of a chemoattractant gradient, whereas cells exposed to a uniform concentration of chemokine do not. Importantly, pseudopodium extension was directional and proceeded in a linear manner, independent of cell body translocation, which allowed differential isolation of this structure from the cell body. Second, cytoskeletal regulatory proteins previously associated with pseudopodia-like structures were present in protein extracts using this fractionation method (FIG. 4). In contrast, nuclear histones associated with the cell body region of polarized cells were not present in the pseudopodial extract. Finally, Rac and Cdc42 activity were increased in extending, but not retracting pseudopodia. Moreover, Rac and Cdc42 activity were necessary for this response as expression of a dominant negative forms of these proteins in cells prevented pseudopodia extension. Rac and Cdc42 are well documented to facilitate actin-based protrusive mechanisms leading to membrane extension and polarity in migratory cells (Ridley et al., 1992; Nobes and Hall, 1999). Together, these findings demonstrate that cells polarize by extending pseudopodia through 3.0-μm pores in the direction of a chemoattractant, and that it is possible to purify these structures for biochemical analysis.

Therefore in another aspect, the invention provides a method of modulating cell migration comprising administration of an agent that regulates actin polymerization or depolymerization. In a related aspect, the invention provides a method of modulating cell migration comprising administration of an agent that regulates myosin contractility. In still another aspect, the invention provides a method of modulating cell migration comprising administration of an agent that regulates adhesive signals that facilitate attachment or detachment of a membrane to an extracellular matrix (ECM).

Therefore, pseudopodia growth requires assembly of a p130Crk-associated substrate (CAS)/c-CrkII (Crk) scaffold, which facilitates translocation and activation of Rac1. Rac1 activation then serves as a positive-feedback loop to maintain CAS/Crk coupling and pseudopodia extension. Conversely, disassembly of this molecular scaffold is critical for export and down regulation of Rac1 activity and induction of pseudopodia retraction. The uncoupling of Crk from CAS during pseudopodium retraction is independent of changes in focal adhesion kinase activity and CAS tyrosine phosphorylation. CAS/Crk is therefore an essential scaffold for Rac1-mediated pseudopodia growth and retraction, and illustrates spatio-temporal segregation of cytoskeletal signals during cell polarization.

The following examples are intended to illustrate but not limit the invention.

### EXAMPLES

As used in the Examples, the sources of cell lines, reagents, and antibodies were as follows. The expression plasmid pUCCAGGS containing full-length as well as mutant forms of the c-Crk cDNA were constructed as described previously (Matsuda et al., 1993; Tanaka et al., 1993). The pEBG expression plasmid containing wild-type CAS or CAS with an in-frame deletion of its substrate domain (CAS-SD) has been described previously (Mayer et al., 1995). Myc-tagged dominant negative Rac1 (N17), dominant positive Rac1 (Q61L), and HA-tagged dominant negative Cdc42 (Cdc42N17) in pcDNA3 have been described previously (Klemke et al., 1998). Antibodies to Crk, CAS, and paxillin, were from Transduction Laboratories. Antibodies to myc (9E10), ERK2, and FAK were from Santa Cruz Biotechnology. Antibodies to phosphotyrosine (monoclonal antibody 4G10), Rho, Rac1, Cdc42, and the GTPase activity kits for these proteins were from Upstate Biotechnology. Goat anti-rabbit and anti-mouse antibodies were from Bio-Rad Laboratories. Antibodies to gelsolin, filamin, the β1 integrin subunit, integrin vβ5, caldesmon, and QCM migration kit were from Chemicon International. Phosphospecific antibodies to FAK tyrosine 397, 576, 577, and PLC-1 tyrosine 783 were from Biosource International. Anti-dynamin II antibodies were provided by Dr. Sandra Schmid (The Scripps Research Institute, La Jolla, Calif.). LPA and anti-vinculin antibodies were from Sigma-Aldrich. 3H-LPA (50 Ci/mmol) was from NEN Life Science Products, Inc. Cell tracker green was from Molecular Probes, Inc. COS-7 cells were from American Type Culture Collection. Mouse NIH3T3 cells were provided by Dr. Tony Hunter (The Salk Institute, La Jolla, Calif.).

### Example 1

Quantitative Pseudopodia Assay

Pseudopodia extension was monitored using a pseudopodia assay kit (ECM 650; Chemicon International) or Costar chambers. Serum-starved cells (75,000) were placed into the upper compartment of a chamber (6.5 μm) equipped with a 3.0-μm porous polycarbonate membrane coated on both sides with an optimal amount of ECM protein (5 μg/ml fibronectin or collagen type I). Cells were allowed to attach and spread on the upper surface of the membrane for 2 h, and then stimulated with LPA (Sigma-Aldrich), insulin, or buffer only, which was placed in the lower chamber to establish a gradient or placed in the upper and lower chamber to form a uniform concentration. Cells were allowed to extend pseudopodia through the pores toward the direction of the gradient for various times. To initiate pseudopodia retraction, the chemoattractant was removed or an equivalent amount of chemoattractant placed in the upper chamber to create a uniform concentration. The cell body on the upper surface was manually removed with a cotton swab and the total pseudopodia protein only on the undersurface was determined using BCA and a microprotein assay system (Pierce Chemical Co.). Additionally, pseudopodia can be stained with 1% crystal violet in 2% ethanol and then the dye eluted with 10% acetic acid, which can be measured in an ELISA plate reader (OD 600) for comparison to a standard curve as previously described (Klemke et al., 1998).

### Example 2

LPA and Insulin as Chemoattractants

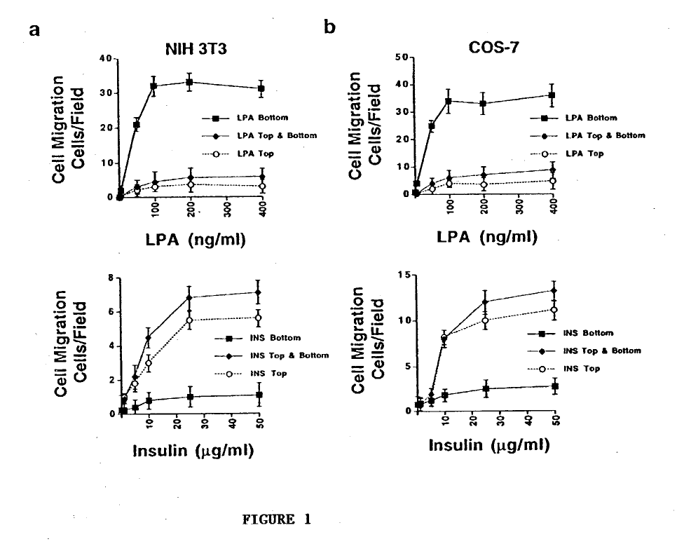

NIH 3T3 cells were examined for cell migration for 3 h in 8.0-μm porous Boyden chambers containing different concentrations of LPA or insulin placed in 1) the bottom, 2) top, or 3) top and bottom compartments. The number of migratory cells per microscopic field (200×) on the underside of the membrane was counted as described in Example 1 above. Additionally, COS-7 cells were examined for cell migration as described above with respect to NIH 3T3 cells. The results are set forth in FIG. 1, where it is seen that LSA is a potent chemoattractant for both cell types. In FIG. 1, each point on the graphs represents the mean±SEM of three triplicate migration chambers of three independent experiments.

### Example 3

Pseudopodia Extension

NIH 3T3 cells were allowed to attach to fibronectin coated 3.0-μm porous membranes for 2 h. Pseudopodia extension was then examined for various times in the absence (NT) or presence of LPA (100 ng/ml) in the bottom, top, or top and bottom compartments. Pseudopodia protein on the underside of the membrane was determined as described in Example 1. Results are set forth in FIG. 2, where each point in the graphs represents the mean±SEM of three triplicate membranes of three independent experiments. COS-7 cells were examined for pseudopodia protrusion in the same manner as for NIH 3T3 cells as described above.

Confocal images of NIH 3T3 pseudopodia protrusion on the undersurface of a 3.0-μm porous membrane in response to LPA (100 ng/ml) as a gradient (LPA bottom) or uniform concentration (LPA top and bottom) were generated. Cells were labeled with cell tracker green and then fixed at the indicated times to visualize protruding pseudopodia on the undersurface of the membrane.

Additionally, diffuision of 3H-LPA (100 ng/ml) from the lower chamber to the upper chamber through a 3.0-μm porous membrane was measured at 60 minutes, 120 minutes and 180 minutes. Each point in the graph in FIG. 2C represents the mean counts per minute (CPM)±SEM of three triplicate chambers of three independent experiments.

COS-7 cells transfected with dominant negative Rac1 (Rac N17), Cdc42 (Cdc42N17), or the empty vector (mock) were examined for pseudopodia formation toward an LPA gradient for 60 min as described in above. (FIG. 2D) An aliquot of cells used for the pseudopodia assay was also lysed and Western blotted for exogenous Rac and Cdc42 expression. RacN17 and Cdc42N17 are myc- and HA-tagged, respectively, and thus show reduced mobility in SDS-PAGE relative to endogenous proteins (Wt). An aliquot of cells transfected as above was examined for cell adhesion to collagen-coated dishes for 30 min in the presence or absence (NT) of LPA (100 ng/ml) as described in Example 1. Results of the above are reported in FIG. 2E as cells attached per microscopic field (200×). Each bar represents the mean±SEM of five fields of three triplicate wells of three independent experiments.

### Example 4

Pseudopedia Retraction

Pseudopodia retraction was studied via the following methods. Pseudopodia extension of NIH 3T3 cells through 3.0-μm pores toward an LPA gradient was examined at 60, 120, 180 and 240 minutes, or the LPA gradient was removed after 60 min and pseudopodia were allowed to retract for the indicated times (LPA Removed). Total pseudopodia protein on the underside of the membrane was determined as described in Example 1.

Pseudopodia were allowed to extend for 60 min (time 0) toward an LPA gradient as described above. The gradient was then removed and pseudopodia were fixed at the indicated times (0, 30, 60, 120 minutes). Cells were labeled with cell tracker green (CTG) to visualize retracting pseudopodia on the undersurface of the membrane by confocal microscopy. Results are set forth in FIG. 2B. Bar, 15 μm.

NIH 3T3 cells labeled with CTG were allowed to extend pseudopodia through 3.0-μm pores toward an LPA gradient for 60 min. The LPA gradient was then removed (time 0) and the pseudopodia allowed to retract for 10, 20 and 30 minutes. Time-lapse images of retracting pseudopodia were taken with a confocal microscope focused at the pore-membrane interface on the undersurface of the polycarbonate membrane.

COS-7 cell pseudopodia extension toward an LPA gradient was determined in the presence or absence (NT) of function-blocking antibodies to vβ5 and β1 integrins (25 μg/ml) for 0, 30, 60, 90 and 120 minutes. Percent pseudopodia growth is the amount of pseudopodia protein on the undersurface of the membrane induced by cells exposed to an LPA gradient relative to cells in the absence of LPA. Results are set forth in FIG. 3B. Retraction of COS-7 cell pseudopodia were determined as described above, with respect to NIH 3T3 cells in the presence or absence (NT) of antibodies to vβ5 and β1 integrins (25 μg/ml) for 0, 30, 60, 90 and 120 minutes. The results are set forth in FIG. 3C. Each point in FIG. 3A, D and E represents the mean±SEM of three triplicate membranes of three independent experiments.

### Example 5

Purification of Pseudopodia, Western Blotting and GTPase Activity Assays

To specifically isolate proteins from growing pseudopodia, 1-1.5×10⁶ cells were induced to form pseudopodia for various times as described above or not treated using a pseudopodia isolation kit (ECM 660; Chemicon International) or chambers from Costar (24 mm 3.0-μm pores). Cells were rinsed in excess cold PBS and either rapidly fixed in 100% ice-cold methanol (Western blotting of whole-cell lysates) or not fixed (for GTPase activity and immunoprecipitation assays). It was found that the protein profile of pseudopodia proteins from fixed and unfixed cells is identical under these conditions as determined by silver staining and SDS-PAGE. Cell bodies on the upper membrane surface were manually removed with cotton swab and pseudopodia on the undersurface scrapped into lysis buffer (100 mM Tris, pH 7.4, 5 mM EDTA, 150 mM NaCl, 1 mM sodium orthovanadate, protease inhibitors (cocktail tablet; Boehringer Mannheim Corp.)) containing the appropriate detergent for Western blotting of whole-cell lysates (1% SDS), CAS/Crk coupling (1% Triton X-100), or GTPase activity assays (Triton X-100 according to the manufacturer's recommendation; UBI). Cell bodies were purified in a similar manner except that pseudopodia on the undersurface were removed and the cell body on the upper surface scraped into lysis buffer and detergent. Retracting pseudopodia were induced for various times by removing or placing chemoattractant in the upper chamber to create a uniform concentration as described above. 25-30 μg of pseudopodia protein is typically obtained from each 24-mm well stimulated with chemokine for 30-60 min. This represents 4-5% of the total cellular protein. Cell culture, transient transfections and efficiency determinations, Western blotting, chemotaxis, and adhesion assays were performed at least three times, and a representative image is shown as previously described (Klemke et al., 1998). Transfection efficiencies of 80% and 70% are routinely obtained in COS-7 and NIH 3T3 cells, respectively.

### Example 6

Biochemical Characterization of Cytoskeletal-Regulatory Proteins in Pseudopodia

NIH 3T3 cells were allowed to extend pseudopodia toward an LPA gradient (100 ng/ml) for 60 min (“growth” in FIG. 4A), or the LPA gradient was removed and pseudopodia allowed to retract for 30 min (“retraction” in FIG. 4A). Proteins (10 μg) isolated from the cell body on the upper membrane surface or pseudopodia (“Pseudo” in FIG. 4A) on the lower membrane surface were resolved by one-dimensional SDS-PAGE and GelCode Staining (Pierce Chemical Co.) as described in Example 1. Total cellular proteins were also isolated from cells attached to culture dishes either not treated (NT) or treated with a uniform concentration of LPA for 60 min (growth control). Cells were also treated with a uniform concentration of LPA for 60 min and then washed and proteins isolated after 30 min of retraction (retraction control). Arrowheads in FIG. 4A indicate nuclear histone proteins which are absent in purified pseudopodia.

Proteins isolated as described above were analyzed by Western blotting using antibodies to the indicated proteins. Whole cell represents total cellular protein isolated from untreated cells attached to fibronectin coated dishes for 90 min. Results are set forth in FIG. 4B.

Proteins prepared from NIH 3T3 cells as described above were also examined for GTP-bound activated Rac and Cdc42 using the p21-binding domain of PAK, which selectively binds Rac- and Cdc42-GTP. Rho-GTP was detected using the Rhotekin Rho binding domain. GTP bound (“active” in FIG. 4C) or total protein (“total” in FIG. 4C) in the corresponding whole-cell lysates was detected by Western blotting as described in Example 5. Densitometry was used to determine the ratio of GTP bound Rac, Cdc42, and Rho to the total GTPase protein present in an aliquot of the same protein lysates used for the activity assay. Fold increase represents the change in GTPase activity in the cell body and pseudopodial fractions relative to basal GTPase activity present in untreated whole cells (NT).

NIH 3T3 cells were either held in suspension for 30 min (“sus” in FIG. 4D) or allowed to attach for 2 h to either fibronectin coated culture dishes, or the upper surface of a 3.0-μm porous membrane coated with fibronectin. Whole cells on culture dishes or pseudopodia in the growth and retraction phase were isolated as described above and analyzed for tyrosine phosphorylation by Western blotting with anti-phosphotyrosine antibodies. Blots treated with ECL reagent were exposed to film for 30 and 90 s. Arrowheads in FIG. 4D indicate proteins with increased phosphotyrosine in purified pseudopodia. The asterisk shows proteins with increased phosphotyrosine in retracting pseudopodia. The total proteins isolated were analyzed by Western blotting with phosphorylation site-specific antibodies to FAK at tyrosine's 397 (autophosphorylation, c-src and PI3K binding sites), 576, and 577 (catalytic activation sites). Blots were stripped and reprobed with antibodies to FAK protein to evaluate the level of FAK protein present in the lysates. Results are shown in FIG. 4E.

Proteins isolated as described above were either analyzed by Western blotting with phosphorylation-site specific antibodies to tyrosine 783 of human PLC-1 or immunoprecipitated with antibodies to CAS and then immunoblotted with antiphosphotyrosine antibodies. NT is total cellular protein isolated from untreated cells attached to fibronectin coated dishes for 120 min. Arrows in FIG. 4F indicate tyrosine phosphorylated proteins of 85 (p85), and 70 kD (p70) that coimmunprecipitate with CAS specifically in growing, but not retracting pseudopodia. Arrowhead shows a tyrosine-phosphorylated protein of 60 kD that coimmunoprecipitates with CAS in the cell body, but not pseudopodia of polarized cells. IgH is the immunoglobulin heavy chain.

### Example 7

CAS/Crk Coupling and Rac Activity in Relation to Pseudopodia

NIH 3T3 cells attached to fibronectin coated 3.0-μm porous membranes were allowed to extend pseudopodia toward a LPA gradient (100 ng/ml) for 60 min (growth), or the LPA gradient was removed and the pseudopodia allowed to retract for 30 min (retraction). Proteins isolated from the cell body or pseudopodia under growth or retraction conditions were prepared as described in Example 6. Total cellular protein (NT) was also isolated from cells attached to culture dishes in the absence of LPA. CAS or Crk was then immunoprecipitated and Western blotted for associated Crk or CAS, respectively. Also, FAK association with CAS was examined in the CAS immunoprecipitates by Western blotting with FAK specific antibodies. The results re set forth in FIG. 5A.

COS-7 cells were transfected with the empty vector (Mock) or the vector encoding either CAS with its substrate domain truncated (CAS-SD) or Crk with a mutated SH2 domain (Crk-SH2). Cells were then examined for pseudopodia extension in response to a LPA gradient (100 ng/ml) or left untreated for 60 min. Pseudopodia protein was determined as described in Example 5. Results are set forth in FIG. 5B. Additionally, COS-7 cells were transfected with the empty vector (Mock) or vectors encoding CAS and Crk. Cells were then examined for Pseudopodia extension in response to a LPA gradient (100 ng/ml) or not treated for 60 min. Pseudopodia protein on the underside of the membrane was determined as described in Example 5. Results are set forth in FIG. 5C. Alternatively, COS-7 cells transfected with the empty vector (Mock) or vectors encoding CAS and Crk were allowed to extend pseudopodia toward an LPA gradient (100 ng/ml) for 60 min (growth), or the LPA gradient was removed and the pseudopodia allowed to retract for 30 min (retraction). Pseudopodia protein was determined as described in Example 5. The bottom panel shows CAS/Crk coupling in purified pseudopodia undergoing growth (60 min) or retraction (60 min) from cells transfected as described above. The results are set forth in FIG. 5D.

Cell body and pseudopodia proteins prepared from cells treated as described above were examined for activated Rac or total Rac protein in whole cell lysates as described in Example 6, above. From the results of the above, as set forth in FIG. 5, it is seen that CAS/Crk coupling and Rac activity regulate pseudopodia growth and retraction.

### Example 8

Effect of Rac on CAS/Crk Complexes in Cells

COS-7 cells were transfected with CAS and Crk along with either myc-tagged dominant negative (RacN17) or dominant positive (RacQ61L) Rac1. Cells were then lysed in detergent (whole cell lysate) and examined for either 1) assembly of CAS/Crk complexes as described in Example 7 or 2) changes in FAK and PLC-1 activity as described in Example 6. Blots were stripped and reprobed with antibodies to FAK and PLC-1 to confirm equal protein loading. The results, as set forth in FIG. 6, show that Rac activity regulates assembly of CAS/Crk complexes in cells.

### Example 9

Time-Lapse Imaging of Retracting Pseudopodia

Cells were labeled with cell tracker green according to the manufacturer's recommendation (Molecular Probes). Pseudopodia retraction was initiated by removing chemoattractant from the lower chamber as described above. The upper compartment (6.5 μm) was then fitted to an Attofluor cell chamber containing a glass coverslip (Molecular Probes) and placed into a stage heater (20/20 Technologies) on a Zeiss Axiovert 100TV microscope. A BioRad 1024 confocal microscope was used to capture time-lapse images of pseudopodia on the undersurface of the membrane every 60 s with a Zeiss 633 Achostigmat lens and LaserSharp software (Bio-Rad Laboratories). QuickTime and Adobe photoshop software were used to prepare time-lapse movies and representative montage images for each time frame assembled for comparison. Immunofluorescent staining and confocal imaging of cells were performed as described (Cheresh et al., 1999).

Although the invention has been described with reference to the above example, it will be understood that modifications and variations are encompassed within the spirit and scope of the invention. Accordingly, the invention is limited only by the following claims.

References:

Allen, W., D. Zicha, and G. Jones. 1998. A role for Cdc42 in macrophage chemotaxis. J. Cell Biol. 141:1147-1157.

Aplin, A., A. Howe, S. Alahari, and R. Juliano. 1998. Signal transduction and signal modulation by cell adhesion receptors: the role of integrins, cadherins, immunoglobulin-cell adhesion molecules, and selectins. Pharm. Rev. 50:197-263.

Azuma, T., W. Witke, T. Stossel, J. Hartwig, and D. Kwiatkowski. 1998. Gelsolin is a downstream effector of Rac for fibroblast motility. EMBO J. 17:1362-1370.

Bailly, M., L. Yan, G. Whitesides, J. Condeelis, and J. Segall. 1998. Regulation of protrusion shape and adhesion to the substratum during chemotactic responses of mammalian carcinoma cells. Exp. Cell Res. 241:285-299.

Cheresh, D., J. Leng, and R. Klemke. 1999. Regulation of cell contraction and membrane ruffling by distinct signals in migratory cells. J. Cell Biol. 146:1107-1116.

Cho, S., and R. Klemke. 2000. Extracellular-regulated kinase activation and CAS/Crk coupling regulate cell migration and suppress apoptosis during invasion of the extracellular matrix. J. Cell Biol. 149:223-236.

Eddy, R., L. Pierini, F. Matsumura, and F. Maxfield. 2000. Ca21-dependent myosin II activation is required for uropod retraction during neutrophil migration. J. Cell Sci. 113:1287-1298.

Etienne-Manneville, S., and A. Hall. 2001. Integrin-mediated activation of Cdc42 controls cell polarity in migrating astrocytes through PKCz. Cell. 106: 489-498.

Fukushima, N., I. Ishii, J. Contos, J. Weiner, and J. Chun. 2001. Lysophospholipid receptors. Annu. Rev. Pharmacl. Toxicol. 41:507-534.

Garton, A., A. Flint, and N. Tonks. 1996. Identification of p130cas as a substrate for the cytosolic protein tyrosine phosphatase PTP-PEST. Mol. Cell Biol. 16:6408-6418.

Helfman, D., E. Levy, C. Berthier, M. Shtutman, D. Riveline, I. Grosheva, A. Lachish-Zalait, M. Elbaum, and A. Bershadsky. 1999. Caldemon inhibits nonmuscle cell contractility and interferes with the formation of focal adhesions. Mol. Biol. Cell. 10:3097-3112.

Jin, T., N. Zhang, Y. Long, C. Parent, P. Deverotes. 2000. Localization of the G protein bg complex in living cells during chemotaxis. Science. 287:1034-1036.

Kain, K., and R. Klemke. 2001. Inhibition of cell migration by Abl family tyrosine kinases through uncoupling of Crk-CAS complexes. J. Biol. Chem. 276:16185-16192.

Kiosses, W., S. Shattil, N. Pampori, and M. Schwartz. 2001. Rac recruits high-affinity integrin avb3 to lamellipodia in endothelial cell migration. Nat. Cell Biol. 3:316-320.

Klemke, R., J. Leng, R. Molander, P. Brooks, K. Vuori, and D. Cheresh. 1998. CAS/Crk coupling serves as a “molecular switch” for induction of cell migration. J. Cell Biol. 140:961-972.

Kraynov, V., C. Chamberlain, G. Bokoch, M. Schwartz, S. Slabaugh, and K. Hahn. 2001. Localized Rac activation dynamics visualized in living cells. Science. 290:333-337.

Lauffenburger, D., and A. Horwitz. 1996. Cell migration: A physically integrated molecular process. Cell. 84:359-369.

Laukaitis, C., D. Webb, K. Donais, and A. Horwitz. 2001. Differential dynamics of a5 integrin, paxillin, and a-actinin during formation and disassembly of adhesions in migrating cells. J. Cell Biol. 153:1427-1440.

Liu, F., D. Hill, and J. Chemoff. 1996. Direct binding of the proline-rich region of protein tyrosine phosphatase 1B to the src homology 3 domain of p130cas. J. Biol. Chem. 271:31290-31295.

Ma, A., A. Richardson, E. Schaefer, and J. Parsons. 2001. Serine phosphorylation of focal adhesion kinase in interphase and mitosis: a possible role in modulating binding to p130cas. Mol. Biol. Cell. 12:1-12.

Manes, S., E. Mira, C. Gomez-Mouton, R. Lacalle, P. Keller, J. Labrador, and C. Martinez-A. 1999. Membrane raft microdomains mediate front-rear polarity in migrating cells. EMBO J. 18:6211-6220.

Matsuda, M., S. Nagata, K. Tanaka, K. Nagashima, and T. Kurata. 1993. Structural requirement of CRK SH2 region for binding to phosphotyrosine containing proteins. J. Biol. Chem. 268:4441-4446.

Mayer, B., H. Hirai, and R. Sakai. 1995. Evidence that SH2 domains promote processive phosphorylation by protein-tyrosine kinases. Curr. Biol. 5:296-305.

McNiven, M., L. Kim, E. Krueger, J. Orth, H. Cao, and T. Wong. 2000. Regulated interactions between dynamin and the actin-binding protein cortactin modulate cell shape. J. Cell Biol. 151:187-198.

Meili, R., C. Ellsworth, S. Lee, T. Reddy, H. Ma, and R. Firtel. 1999. Chemoattractant-mediated transient activation and membrane localization of Akt/PKB is required for efficient chemotaxis to cAMP in Dictyostelium. EMBO J. 18:2092-2105.

Nobes, C., and A. Hall. 1999. Rho GTPases control polarity, protrusion, and adhesion during cell movement. J. Cell Biol. 144:1235-1244.

Ohta, Y., N. Suzuki, S. Nakamura, J. Hartwig, and T. Stossel. 1999. The small GTPases Ra1A targets filamin to induce filopodia. Proc. Natl. Acad. Sci. 96:2122-2128.

Parent, C., and P. Devreotes. 1999. A cell's sense of direction. Science. 284:765-770.

Parent, C., B. Blacklock, W. Froehlich, D. Murphy, and P. Devreotes. 1998. G protein signaling events are activated at the leading edge of chemotactic cells. Cell. 95:81-91.

Ridley, A., H. Paterson, C. Johnston, D. Diekman, and A. Hall. 1992. The small GTP-binding protein Rac regulates growth factor-induced membrane ruffling. Cell. 70:401-410.

Schmidt, C., A. Horwitz, D. Lauffenburger, and M. Sheetz. 1993. Integrin cytoskeletal interactions in migrating fibroblasts are dynamic and asymmetrical, and regulated. J. Cell Biol. 123:977-991.

Schmitz, A., E. Govek, B. Bottner, and L. Van Aelst. 2000. Rho GTPases: Signaling, migration, and invasion. Exp. Cell Res. 261:1-12.

Servant, G., O. Weiner, P. Herzmark, T. Balla, J. Sedat, and H. Bourne. 2000. Polarization of chemoattractant receptor signaling during neutrophil chemotaxis. Science. 287:1037-1040.

Smilenov, L., A. Mikhailov, Jr. R. Pelham, E. Marcantonio, and G. Gundersen. 1999. Focal adhesion motility revealed in stationary fibroblasts. Science. 286:1172-1174.

Tachibana, K., T. Urano, H. Fujita, Y. Ohashi, K. Kamiguchi, S. Iwata, H. Hirai, and C. Morimoto. 1997. Tyrosine phosphorylation of Crk-associated substrates by focal adhesion kinase. J. Biol. Chem. 272:29083-29090.

Tanaka, S., S. Hattori, T. Kurata, K. Nagashima, Y. Fukui, S. Nakamura, and M. Matsuda. 1993. Both the SH2 and SH3 domains of Crk protein required for neuronal differentiation of PC12 cells. Mol. Cell. Biol. 13:4409-4415.

Vuori, K., H. Hirai, S. Aizawa, and E. Ruoslahti. 1996. Induction of p130CAS signaling complex formation upon integrin-mediated cell adhesion: a role for src family kinases. Mol. Cell. Biol. 16:2606-2613.

What is claimed is:

1. A method of isolating a pseudopodium of a cell, comprising:
a) placing a population of cells on a porous membrane;
b) stimulating the cells with a chemoattractant on the opposite side of the membrane from where the cells are placed, such that at least one cell is stimulated to extend a pseudopodium through the pores of the porous membrane, leaving a cell body on the porous membrane; and
c) removal of the pseudopodia extending through the pores of the porous membrane.

2. The method of claim 1, further comprising identifying the proteins of the removed pseudopodia.

3. The method of claim 1, further comprising removal of the cell bodies remaining on the surface of the porous membrane.

4. The method of claim 3, further comprising identifying the proteins of the removed cell bodies.

5. The method of claim 1, wherein the porous membrane is a porous polycarbonate membrane.

6. The method of claim 1, wherein the chemoattractant is lysophosphatidic acid (LPA).

7. The method of claim 1, wherein removal of the pseudopodia extending through the pores of the porous membrane is performed with a cotton swab.

8. A pseudopodium isolated by the method of claim 1.

9. A method of inducing extension of a pseudopodia from a cell, comprising:
a) placing a population of cells on a porous membrane; and
b) stimulating the cells with a chemoattractant on the opposite side of the membrane from where the cells are placed, such that at least one cell is stimulated to extend a pseudopodium.

10. The method of claim 9, wherein the chemoattractant is lysophosphatidic acid (LPA).

11. A method of inducing retraction of a pseudopodia from a cell, comprising:
a) placing a population of cells on a porous membrane; and
b) stimulating the cells with a chemoattractant on both sides of the membrane, such that at least one cell is stimulated to retract a pseudopodium.

12. The method of claim 11, wherein the chemoattractant is lysophosphatidic acid (LPA).

13. A method of identifying an agent effective in inducing extension or retraction of a pseudopodium comprising:
a) placing a population of cells on a porous membrane;
b) measuring the number of cells that have a pseudopodium extended through the pores of the porous membrane, leaving a cell body on the porous membrane;
c) stimulating cells with an agent on the opposite side of the membrane from where the cells are placed; and
d) measuring the number of cells that are stimulated to extend a pseudopodium through the pores of the porous membrane,
wherein an increase in the number of cells extending a pseudopodium from step b) to step d) is indicative of an agent effective in inducing extension of a pseudopodium and a decrease in the number of cells extending a pseudopodium from step b) to step d) is indicative of an agent effective in inducing retraction of a pseudopodium.

14. The method of claim 13, further comprising isolating the extended pseudopodia and determining the proteins contained therein.

15. The method of claim 13, wherein the agent increases the number of cells extending a pseudopodium.

16. The method of claim 15, wherein the agent stimulates at least about 90% of the cells to extend a pseudopodium.

17. The method of claim 13, wherein the agent decreases the number of cells extending a pseudopodium.

18. A method of modulating cell migration comprising administering an effective amount of an agent identified by the method of claim 13, wherein the agent is effective in inducing extension or retraction of a pseudopodium.

19. A method of modulating a cell migration-associated process comprising administering an effective amount of an agent identified by the method of claim 13, wherein the agent is effective in modulating the cell migration-associated process.

20. The method of claim 19, wherein the cell migration-associated process is selected from cell and tissue development, wound healing, immune responses, angiogenesis, embryonic development, metastases, neuronal regeneration, stem cell migration, and inflammation.

21. A method of modulating cell migration comprising administration of a composition that induces extension or retraction of a pseudopodium, thereby modulating cell migration.

22. A method of identifying the state of a pseudopodium comprising measuring the level of Rac or Cdc42 in an isolated pseudopodium, wherein an increase in Rac or Cdc42 activity in the pseudopodium is indicative a state of extension of the pseudopodium and wherein a decrease of Rac or Cdc42 activity in the pseudopodium is indicative of a state of retraction of the pseudopodium.

23. A method of modulating cell migration comprising administration of an agent that regulates actin polymerization or depolymerization.

24. A method of modulating cell migration comprising administration of an agent that regulates myosin contractility.

25. A method of modulating cell migration comprising administration of an agent that regulates adhesive signals that facilitate attachment or detachment of a membrane to an extracellular matrix (ECM).

26. A method of identifying proteins specifically expressed in the pseudopodium or specifically expressed in the cell body of a cell during a cell migration-associated process comprising:
a) identifying the proteins expressed in the pseudopodium during migration;
b) identifying the proteins expressed in the cell body during migration; and
c) comparing the proteins expressed in the pseudopodium to the proteins present in the cell body,
wherein the comparing allows identification of proteins similar to the pseudopodium and cell body and wherein a difference in expression is indicative of proteins specifically expressed in the pseudopodium or the cell body.

27. The method of claim 26, further comprising
i) placing a population of cells on a porous membrane;
ii) stimulating the cells with a chemoattractant on the opposite side of the membrane from where the cells are placed, such that at least one cell is stimulated to extend a pseudopodium through the pores of the porous membrane, leaving a cell body on the porous membrane; and
iii) removal of the pseudopodia extending through the pores of the porous membrane
prior to identifying the proteins in the pseudopodium.

28. The method of claim 27, further comprising
iv) removal of the cell bodies remaining on the porous membrane
prior to identifying the proteins in the cell body.

29. The method of claim 26, wherein the comparing is performed by brute force mass spectrometry.

30. The method of claim 26, wherein the comparing is performed by two dimensional gel electrophoresis.

31. A method of modulating a cell migration-associated process comprising administering an effective amount of an agent targeted to modulate expression of a protein identified by the method of claim 26.

32. The method of claim 31, wherein the cell migration-associated process is selected from cell and tissue development, wound healing, immune responses, angiogenesis, embryonic development, metastases, neuronal regeneration, stem cell migration, and inflammation.

33. A method of identifying polynucleotides specifically expressed in the pseudopodium or specifically expressed in the cell body of a cell during a cell migration-associated process comprising:
a) identifying the polynucleotides expressed in the pseudopodium during migration;
b) identifying the polynucleotides expressed in the cell body during migration; and
c) comparing the expression in the pseudopodium to the expression in the cell body,
wherein the comparing allows identification of expression of polynucleotides similar to the pseudopodium and cell body and wherein a difference in expression is indicative of polynucleotides specifically expressed in the pseudopodium or the cell body.
